(19) United States
(12) Patent Application Publication
(10) Pub. No.: US 2026/0001822 A1
(43) Pub. Date: Jan. 1, 2026

(54) SYNTHESIS OF ALKYL CYCLOHEXANES

(71) Applicant: Chevron Phillips Chemical Company LP, The Woodlands, TX (US)

(72) Inventors: Brooke Small, Kingwood, TX (US); Thomas J. Malinski, Porter, TX (US); Jeffery C. Gee, Kingwood, TX (US)

(21) Appl. No.: 18/758,072

(22) Filed: Jun. 28, 2024

(51) Int. Cl. C07C 2/02; B01J 27/125; C09K 5/10; F28D 1/02

(52) U.S. Cl. CPC C07C 2/02; B01J 27/125; C09K 5/10; F28D 1/0206; C07C 2527/125

(57) ABSTRACT

Production of C₁₆ to C₃₀ saturated hydrocarbon by alkylating a reactant aromatic hydrocarbon with a C₆ to C₂₄ olefin to produce alkylation product comprising a C₁₆ to C₃₀ alkyl aromatic hydrocarbon and subsequent hydrogenation of the alkylation product to produce hydrogenated product comprising a C₁₆ to C₃₀ saturated hydrocarbon. The C₁₆ to C₃₀ alkyl aromatic hydrocarbon comprises an aromatic C₆ ring and one or more C₆ to C₂₄ alkyl groups attached thereto. The C₁₆ to C₃₀ saturated hydrocarbon comprises a saturated C₆ ring and one or more C₆ to C₂₄ alkyl groups attached thereto.

### FIELD OF THE DISCLOSURE

The present disclosure generally relates to saturated hydrocarbons, and more particularly to the synthesis of alkyl-substituted cyclohexanes achieved by alkylating aromatic hydrocarbons with olefins and hydrogenation of the resultant alkylation product.

### BACKGROUND

Cooling fluids play a significant role in various industrial applications such as heat transfer, lubrication, and refrigeration. The use of synthetic oils as cooling fluids has gained attention due to their superior thermal stability and lubricating properties. Poly alpha olefins (PAOs) are a type of synthetic oil that has gained significant popularity as a cooling fluid due to their unique properties.

PAOs are synthesized by the polymerization of alpha olefins, which are unsaturated hydrocarbons with a carbon-carbon double bond at a terminal carbon. They have relatively high viscosity indexes, which means they can maintain their viscosity at different temperatures. This makes them suitable for use in cooling applications where traditional mineral oils would break down. PAOs also have excellent lubricating properties, making them a highly satisfactory choice for cooling systems that require both cooling and lubrication. Their low coefficient of friction reduces wear on moving parts.

While PAOs are highly satisfactory use as cooling fluids, there is a need for improved cooling fluids that exhibit improved densities, viscosities, viscosity indexes, and/or pour points.

### SUMMARY

Disclosed is a process that includes: alkylating i) a reactant aromatic hydrocarbon including benzene, toluene, xylene, or combinations thereof with ii) a C₆ to C₂₄ olefin, to produce an alkylation product including a C₁₆ to C₃₀ alkyl aromatic hydrocarbon, wherein the C₁₆ to C₃₀ alkyl aromatic hydrocarbon includes an aromatic C₆ ring and one or more C₆ to C₂₄ alkyl groups attached to the aromatic C₆ ring; and hydrogenating the C₁₆ to C₃₀ alkyl aromatic hydrocarbon to produce a hydrogenated product including a C₁₆ to C₃₀ saturated hydrocarbon, wherein the C₁₆ to C₃₀ saturated hydrocarbon includes a saturated C₆ ring and one or more C₆ to C₂₄ alkyl groups attached to the saturated C₆ ring.

Disclosed is an alkyl cyclohexane that includes a C₁₆ to C₃₀ saturated hydrocarbon, wherein the C₁₆ to C₃₀ saturated hydrocarbon includes a saturated C₆ ring and one or more C₆ to C₂₄ alkyl groups attached to the saturated C₆ ring.

Disclosed is an immersion coolant comprising an alkyl cyclohexane comprising a C₁₆ to C₃₀ saturated hydrocarbon.

Disclosed is process comprising cooling an equipment with an immersion coolant comprising the alkyl cyclohexane comprising a C₁₆ to C₃₀ saturated hydrocarbon.

Other technical features may be readily apparent to one skilled in the art from the following figures, descriptions and claims.

### BRIEF DESCRIPTION OF THE DRAWINGS

For a more complete understanding of this disclosure, reference is now made to the following description, taken in conjunction with the accompanying drawings, in which:

FIG. 1 is a graph of thermal conductivity vs temperature for the hydrogenated product.

FIG. 2 is a graph of specific heat vs temperature for the hydrogenated product.

### DETAILED DESCRIPTION

To define more clearly the terms used herein, the following definitions are provided. Unless otherwise indicated, the following definitions are applicable to this disclosure. If a term is used in this disclosure, but is not specifically defined herein, the definition from the IUPAC Compendium of Chemical Terminology, 2nd Ed (1997) can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Groups of elements of the Periodic Table are indicated using the numbering scheme indicated in the version of the Periodic Table of elements published in Chemical and Engineering News, 63(5), 27, 1985. In some instances, a group of elements can be indicated using a common name assigned to the group; for example, alkali metals for Group 1 elements, alkaline earth metals (or alkaline metals) for Group 2 elements, transition metals for Groups 3-12 elements, and halogens for Group 17 elements.

Regarding claim transitional terms or phrases, the transitional term “comprising,” which is synonymous with “including,” “containing,” “having,” or “characterized by,” is inclusive or open-ended and does not exclude additional, unrecited elements or method steps. The transitional phrase “consisting of” excludes any element, step, or ingredient not specified in the claim. The transitional phrase “consisting of” limits the scope of a claim to the specified materials or steps and those that do not materially affect the basic and novel characteristic(s) of the claimed invention. The term “consisting essentially of” occupies a middle ground between closed terms like “consisting of” and fully open terms like “comprising.” Absent an indication to the contrary, when describing a compound or composition, “consisting essentially of” is not to be construed as “comprising,” but is intended to describe the recited component that includes materials which do not significantly alter the composition or method to which the term is applied. For example, a composition consisting essentially of a material A can include impurities typically present in a commercially produced or commercially available sample of the recited compound or composition. When a claim includes different features and/or feature classes (for example, a method step, composition features, and/or product features, among other possibilities), the transitional terms comprising, consisting essentially of, and consisting of apply only to the feature class which is utilized and it is possible to have different transitional terms or phrases utilized with different features within a claim. For example, a method can comprise several recited steps (and other non-recited steps), but utilize a catalyst system preparation consisting of specific steps, or alternatively, consisting essentially of specific steps, but utilize a catalyst system comprising recited components and other non-recited components.

While compositions and methods are described in terms of “comprising” (or other broad term) various components and/or steps, the compositions and methods can also be described using narrower terms, such as “consist essentially of” or “consist of” the various components and/or steps.

The terms “a,” “an,” and “the” are intended, unless specifically indicated otherwise, to include plural alternatives, e.g., at least one.

For any compound disclosed herein, the general structure or name presented is also intended to encompass all structural isomers, conformational isomers, and stereoisomers that can arise from a particular set of substituents, unless indicated otherwise. Thus, a general reference to a compound includes all structural isomers, unless explicitly indicated otherwise; e.g., a general reference to hexene includes n-hexene, 2-methyl-pentene, 3-methyl-pentene, 3-isopropyl-propene, 2,3-dimethyl-butene, and 2-diethyl-ethene, and a general reference to heptane include n-heptene, 2-methyl-hexene, 3-methyl-hexene, 4-methyl-hexene, 4-isopropyl-propene, 2,3-dimethyl-pentene, 2,4-dimethyl-propene, 3,4-dimethyl-pentene, 2-ethyl-pentene, 2-isopentyl-ethene, and 2-ethyl-3-methyl-butene while a general reference to a butyl group includes an n-butyl group, a sec-butyl group, an iso-butyl group, and a tert-butyl group. One of ordinary skill in the art will understand how the same reasoning is applied to the C₈-24 structural isomers of octene, nonene, decene, undecene, dodecene, tridecene, tetradecene, pentadecene, hexadecene, heptadecene, octadecene, nondecene, eicosene, heneicosene, docosene, tricosene, and tetracosene Additionally, the reference to a general structure or name encompasses all enantiomers, diastereomers, and other optical isomers, whether in enantiomeric or racemic forms, as well as mixtures of stereoisomers, as the context permits or requires. For any formula or name that is presented, any general formula or name presented also encompasses all conformational isomers, regioisomers, and stereoisomers that can arise from a particular set of substituents.

A chemical “group” is described according to how that group is formally derived from a reference or “parent” compound, for example, by the number of hydrogen atoms formally removed from the parent compound to generate the group, even if that group is not literally synthesized in this manner. By way of example, an “alkyl group” can formally be derived by removing one hydrogen atom from an alkane, while an “alkylene group” can formally be derived by removing two hydrogen atoms from an alkane. Moreover, a more general term can be used to encompass a variety of groups that formally are derived by removing any number (“one or more”) of hydrogen atoms from a parent compound, which in this example can be described as an “alkane group,” and which encompasses an “alkyl group,” an “alkylene group,” and materials having three or more hydrogens atoms, as necessary for the situation, removed from the alkane. Throughout, the disclosure of a substituent, ligand, or other chemical moiety that can constitute a particular “group” implies that the well-known rules of chemical structure and bonding are followed when that group is employed as described. When describing a group as being “derived by,” “derived from,” “formed by,” or “formed from,” such terms are used in a formal sense and are not intended to reflect any specific synthetic methods or procedures, unless specified otherwise or the context requires otherwise.

The term “hydrocarbon” whenever used in this specification and claims refers to a compound containing only carbon and hydrogen. Other identifiers can be utilized to indicate the presence of groups in the hydrocarbon (e.g., halogenated hydrocarbon indicates the presence of one or more halogen atoms replacing an equivalent number of hydrogen atoms in the hydrocarbon). The term “hydrocarbyl group” is used herein in accordance with the definition specified by IUPAC: a univalent group formed by removing a hydrogen atom from a hydrocarbon. Non-limiting examples of hydrocarbyl groups include ethyl, phenyl, tolyl, propenyl, and the like. Similarly, a “hydrocarbylene group” refers to a group formed by removing two hydrogen atoms from a hydrocarbon, either two hydrogen atoms from one carbon atom or one hydrogen atom from each of two different carbon atoms. Therefore, in accordance with the terminology used herein, a “hydrocarbon group” refers to a generalized group formed by removing one or more hydrogen atoms (as necessary for the group) from a hydrocarbon. A “hydrocarbyl group,” “hydrocarbylene group,” and “hydrocarbon group” can be acyclic or cyclic groups, and/or can be linear or branched. A “hydrocarbyl group,” “hydrocarbylene group,” and “hydrocarbon group” can include rings, ring systems, aromatic rings, and aromatic ring systems, which contain only carbon and hydrogen. “Hydrocarbyl groups,” “hydrocarbylene groups,” and “hydrocarbon groups” include, by way of example, aryl, arylene, arene, alkyl, alkylene, alkane, cycloalkyl, cycloalkylene, cycloalkane, aralkyl, aralkylene, and aralkane groups, among other groups, as members.

The term “alkane” whenever used in this specification and claims refers to a saturated hydrocarbon compound. Other identifiers can be utilized to indicate the presence of groups in the alkane (e.g., halogenated alkane indicates the presence of one or more halogen atoms replacing an equivalent number of hydrogen atoms in the alkane). The term “alkyl group” is used herein in accordance with the definition specified by IUPAC: a univalent group formed by removing a hydrogen atom from an alkane. Similarly, an “alkylene group” refers to a group formed by removing two hydrogen atoms from an alkane (either two hydrogen atoms from one carbon atom or one hydrogen atom from two different carbon atoms). An “alkane group” is a general term that refers to a group formed by removing one or more hydrogen atoms (as necessary for the group) from an alkane. An “alkyl group,” “alkylene group,” and “alkane group” can be acyclic or cyclic groups, and/or can be linear or branched unless otherwise specified. Primary, secondary, and tertiary alkyl group are derived by removal of a hydrogen atom from a primary, secondary, and tertiary carbon atom, respectively, of an alkane. The n-alkyl group can be derived by removal of a hydrogen atom from a terminal carbon atom of a linear alkane.

An aliphatic compound is an acyclic or cyclic, saturated or unsaturated carbon compound, excluding aromatic compounds. Thus, an aliphatic compound is an acyclic or cyclic, saturated or unsaturated carbon compound, excluding aromatic compounds; that is, an aliphatic compound is a non-aromatic organic compound. An “aliphatic group” is a generalized group formed by removing one or more hydrogen atoms (as necessary for the group) from a carbon atom of an aliphatic compound. Thus, an aliphatic compound is an acyclic or cyclic, saturated or unsaturated carbon compound, excluding aromatic compounds. That is, an aliphatic compound is a non-aromatic organic compound. Aliphatic compounds and therefore aliphatic groups can contain organic functional group(s) and/or atom(s) other than carbon and hydrogen.

The term “substituted” when used to describe a compound or group, for example, when referring to a substituted analog of a particular compound or group, is intended to describe any non-hydrogen moiety that formally replaces a hydrogen in that group, and is intended to be non-limiting. A group or groups can also be referred to herein as “unsubstituted” or by equivalent terms, such as “non-substituted,” which refers to the original group in which a non-hydrogen moiety does not replace a hydrogen within that group. “Substituted” is intended to be non-limiting and include inorganic substituents or organic substituents.

The term “olefin” whenever used in this specification and claims refers to hydrocarbons that have at least one carbon-carbon double bond that is not part of an aromatic ring or an aromatic ring system. The term “olefin” includes aliphatic and aromatic, cyclic and acyclic, and/or linear and branched hydrocarbons having at least one carbon-carbon double bond that is not part of an aromatic ring or ring system unless specifically stated otherwise. Olefins having only one, only two, only three, etc., carbon-carbon double bonds can be identified by use of the term “mono,” “di,” “tri,” etc., within the name of the olefin. The olefins can be further identified by the position of the carbon-carbon double bond(s).

The term “alkene” whenever used in this specification and claims refers to a linear or branched aliphatic hydrocarbon olefin that has one or more carbon-carbon double bonds. Alkenes having only one, only two, only three, etc., such multiple bonds can be identified by use of the term “mono,” “di,” “tri,” etc., within the name. For example, alkamonoenes, alkadienes, and alkatrienes refer to linear or branched acyclic hydrocarbon olefins having only one carbon-carbon double bond (acyclic having a general formula of CnH2n), only two carbon-carbon double bonds (acyclic having a general formula of CnH2n-2), and only three carbon-carbon double bonds (acyclic having a general formula of CnH2n-4), respectively. Alkenes can be further identified by the position of the carbon-carbon double bond(s). Other identifiers can be utilized to indicate the presence or absence of groups within an alkene. For example, a haloalkene refers to an alkene having one or more hydrogen atoms replaced with a halogen atom.

The term “alpha olefin” as used in this specification and claims refers to an olefin that has a carbon-carbon double bond between the first and second carbon atoms of the longest contiguous chain of carbon atoms. The term “alpha olefin” includes linear and branched alpha olefins unless expressly stated otherwise. In the case of branched alpha olefins, a branch can be at the 2 position (a vinylidene) and/or the 3 position or higher with respect to the olefin double bond. The term “vinylidene” whenever used in this specification and claims refers to an alpha olefin having a branch at the 2 position with respect to the olefin double bond. By itself, the term “alpha olefin” does not indicate the presence or absence of other carbon-carbon double bonds unless explicitly indicated.

The term “normal alpha olefin” whenever used in this specification and claims refers to a linear aliphatic mono-olefin having a carbon-carbon double bond between the first and second carbon atoms. It is noted that “normal alpha olefin” is not synonymous with “linear alpha olefin” as the term “linear alpha olefin” can include linear olefinic compounds having a double bond between the first and second carbon atoms.

The terms “room temperature” or “ambient temperature” are used herein to describe any temperature from 15° C. to 35° C. wherein no external heat or cooling source is directly applied to the reaction vessel. Accordingly, the terms “room temperature” and “ambient temperature” encompass the individual temperatures and any and all ranges, subranges, and combinations of subranges of temperatures from 15° C. to 35° C. wherein no external heating or cooling source is directly applied to the reaction vessel. The term “atmospheric pressure” is used herein to describe an earth air pressure wherein no external pressure modifying means is utilized. Generally, unless practiced at extreme earth altitudes, “atmospheric pressure” is about 1 atmosphere (alternatively, about 14.7 psi or about 101 kPa).

Features within this disclosure that are provided as a minimum value can be alternatively stated as “at least” or “greater than or equal to” any recited minimum value for the feature disclosed herein. Features within this disclosure that are provided as a maximum value can be alternatively stated as “less than or equal to” or “below” any recited maximum value for the feature disclosed herein.

Within this disclosure, the normal rules of organic nomenclature will prevail. For instance, when referencing substituted compounds or groups, references to substitution patterns are taken to indicate that the indicated group(s) is (are) located at the indicated position and that all other non-indicated positions are hydrogen. For example, reference to a 4-substituted phenyl group indicates that there is a non-hydrogen substituent located at the 4 position and hydrogens located at the 2, 3, 5, and 6 positions. By way of another example, reference to a 3-substituted naphth-2-yl indicates that there is a non-hydrogen substituent located at the 3 position and hydrogens located at the 1, 4, 5, 6, 7, and 8 positions. References to compounds or groups having substitutions at positions in addition to the indicated position will be referenced using comprising or some other alternative language. For example, a reference to a phenyl group comprising a substituent at the 4 position refers to a phenyl group having a non-hydrogen substituent group at the 4 position and hydrogen or any non-hydrogen group at the 2, 3, 5, and 6 positions.

Use of the term “optionally” with respect to any element of a claim is intended to mean that the subject element is required, or alternatively, is not required. Both alternatives are intended to be within the scope of the claim.

Unless otherwise specified, any carbon-containing group for which the number of carbon atoms is not specified can have, according to proper chemical practice, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, or any range or combination of ranges between these values. For example, unless otherwise specified, any carbon-containing group can have from 1 to 30 carbon atoms, from 1 to 25 carbon atoms, from 1 to 20 carbon atoms, from 1 to 15 carbon atoms, from 1 to 10 carbon atoms, or from 1 to 5 carbon atoms. Moreover, other identifiers or qualifying terms can be utilized to indicate the presence or absence of a particular substituent, a particular regiochemistry and/or stereochemistry, or the presence or absence of a branched underlying structure or backbone.

Processes and/or methods described herein utilize steps, features, and compounds which are independently described herein. The process and methods described herein may or may not utilize step identifiers (e.g., 1), 2), etc., a), b), etc., or i), ii), etc.), features (e.g., 1), 2), etc., a), b), etc., or i), ii), etc.), and/or compound identifiers (e.g., first, second, etc.). However, it should be noted that processes and/or methods described herein can have multiple steps, features (e.g., reagent ratios, formation conditions, among other considerations), and/or multiple compounds having the same general descriptor. Consequently, it should be noted that the processes and/or methods described herein can be modified to use an appropriate step or feature identifier (e.g., 1), 2), etc., a), b), etc., or i), ii), etc.) and/or compound identifier (e.g., first, second, etc.) regardless of step, feature, and/or compound identifier utilized in a particular aspect and/or embodiment described herein and that step or feature identifiers can be added and/or modified to indicate individual different steps/features/compounds utilized within the process and/or methods without detracting from the general disclosure.

Embodiments disclosed herein can provide the materials listed as suitable for satisfying a particular feature of the embodiment delimited by the term “or.” For example, a particular feature of the disclosed subject matter can be disclosed as follows: Feature X can be A, B, or C. It is also contemplated that for each feature the statement can also be phrased as a listing of alternatives such that the statement “Feature X is A, alternatively B, or alternatively C” is also an embodiment of the present disclosure whether or not the statement is explicitly recited.

The weight percent compositional aspects of the various compositions described herein (e.g., the weight percent of one or more compounds present in a composition) can be determined by gas chromatography (GC), gas chromatography-mass spectroscopy (GC-MS), Raman spectroscopy, nuclear magnetic resonance (NMR) spectroscopy, or any other suitable analytical method known to those of skill in the art. For example, unless otherwise indicated, the weight percent compositional aspects of the various compositions described herein (e.g., the weight percent of the various olefins and saturated hydrocarbons such as C₂₀ olefin dimer and saturated C₂₀ hydrocarbons present in the compositions such as the crude, light fraction, intermediate fraction, heavy faction, etc.) can be determined using a gas chromatograph with a flame ionization detector (GC-FID) detector based on the total GC peak areas (as described herein) and reported as gas chromatography (GC) area percent (GC area %), which is a common analytical technique for compositions comprising sulfur-containing compounds. While not wishing to be bound by this theory, it is believed that the amount in area % is very similar to the amount in weight percent (wt %), and these respective amounts need not be exactly equivalent or interchangeable to be understood by a person of ordinary skill.

It has been found that an alkyl cyclohexane product can be made by alkylating a composition comprising one or more aromatic hydrocarbons with a composition comprising one or more olefins to produce an alkylation product comprising an aromatic hydrocarbon that comprises an aromatic ring and one or more alkyl groups attached to the aromatic ring. The alkylation product is then hydrogenated to produce a hydrogenated product. The hydrogenated product comprises a saturated hydrocarbon that comprises a saturated ring and one or more alkyl groups attached to the saturated ring.

### One or More Aromatic Hydrocarbons

The one or more aromatic hydrocarbons can be chosen from benzene (represented by Structure 1), toluene (represented by Structure 2), o-xylene (represented by Structure 3), m-xylene (represented by Structure 4), p-xylene (represented by Structure 5), or combinations thereof.

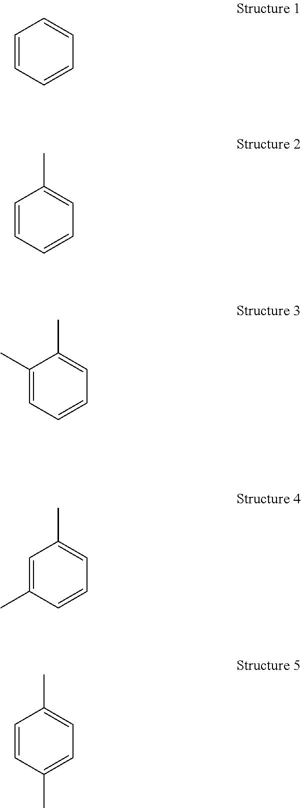

In some aspects, the aromatic hydrocarbon is m-xylene.

### Olefins

The one or more olefins can be chosen from C₆ to C₂₄ branched or straight olefins and combinations thereof. In additional aspects, the C₆ to C₂₄ olefin is/are an internal olefin(s). In aspects, some or all of the C₆ to C₂₄ branched or straight olefin is a C₆ straight or branched olefin, including hexene, pentene, octene, nonene, decene, undecene, dodecene, tridecene, tetradecene, pentadecene, hexadecene, heptadecene, octadecene, nondecene, heneicosene, docosene, tricosene, and tetracosane, wherein the terminal carbon is double bonded to the adjacent carbon or the carbon double bond is between two non-terminal carbons.

In aspects, some or all of the one or more C₆ to C₂₄ branched or straight olefins comprise C₆ to C₁₁ branched or straight olefins.

In aspects, some or all of the C₆ to C₂₄ straight or branched olefins are C₁₂ to C₂₄ straight or branched olefins.

In aspects, some or all of the one or more C₆ to C₂₄ branched or straight olefins is 1-tetradecene.

In aspects, any of the processes described herein can optionally include an additional step of oligomerizing one or more precursor olefin monomer to form the one or more C₆ to C₂₄ branched or straight olefins.

Any composition comprising one or more C₆ to C₂₄ branched or straight olefins of the type described herein can be used, for example a composition obtained from a commercial petroleum refining or petrochemical process. Such compositions can comprise other olefins in addition to the C₆ to C₂₄ branched or straight olefins of the type described herein, for example branched or straight olefins having less than 6 carbon atoms or more than 24 carbon atoms.

In another embodiment, the composition comprising one or more C₆ to C₂₄ branched or straight olefins can be produced by purifying an effluent stream obtained from a 1-hexene production process tock, for example by distillation of the effluent stream obtained from an olefin production process.

### Alkylation Conditions

Any suitable reactor or vessel can be used to perform the contacting step to form the alkylation product via alkylating reactions. Non-limiting examples of a reactor or vessel can include a flow reactor, a continuous reactor, a packed tube, and a stirred tank reactor, including more than one reactor in series or in parallel, and including any combination of reactor types and arrangements.

The alkylating step can be conducted at a variety of temperatures, pressures, and reaction times. Contact between the comprising one or more C₆ to C₂₄ branched or straight olefins and the one or more aromatic hydrocarbons can generally include initial contact between the aromatic hydrocarbon and an optional catalyst, and subsequent contact between the one or more C₆ to C₂₄ branched or straight olefins with the combined one or more aromatic hydrocarbons/optional catalyst. For instance, the temperature at which the one or more aromatic hydrocarbons and the one or more C₆ to C₂₄ branched or straight olefins are initially contacted can be the same as, or different from, the temperature at which the alkylation product is formed. As an illustrative example, in the alkylation step, the one or more C₆ to C₂₄ branched or straight olefins and the catalyst can be contacted and combined initially at temperature T1 and, after this initial contact, the temperature may rise, due to an exotherm from the alkylation reaction, to a temperature T2 higher than T1. Likewise, the pressure in the contacting step can be at pressure P1 for initial contact and P2 for subsequent contact. The contact time can be referred to as the reaction time.

In aspects, the alkylation step can be conducted at a temperature in a range of from 40° C. to 80° C.; alternatively, from 50° C. to 70° C. at a minimum temperature of 40, 41, 42, 43, 44, 45, 46, 47, 48, 49, or 50° C. and at a maximum temperature of 80, 79, 78, 77, 76, 75, 74, 73, 72, 71, or 70° C. In aspects, the alkylation step can be conducted at a temperature in a range from any minimum temperature disclosed herein to any maximum temperature disclosed herein. These temperature ranges also are meant to encompass circumstances where the alkylation step can be conducted at a series of different temperatures, instead of at a single fixed temperature, falling within the respective temperature ranges.

In aspects, the alkylation step can be conducted at any pressure suitable for an alkylation reaction of the type disclosed herein. For example, the reaction pressure can be at atmospheric pressure alkylation carried out in an open reaction vessel or at an autogenous pressure for alkylation carried out in a seal reaction vessel.

In aspects, the alkylation step can be conducted at a reaction time in a range of from 1 hr to 48 hrs; alternatively, from 1 hr to 24 hrs; alternatively, from 1 hr to 12 hrs; alternatively, from 2 hrs to 10 hrs; alternatively, from 2 hrs to 7 hrs.

In aspects, a mole ratio of the reactant aromatic hydrocarbon to the C₆ to C₂₄ straight or branched olefin is in a range of from 1:1 to excess:1, alternatively, from 1:1 to 2:1, alternatively, from 1:1 to 3:1, alternatively, from 1:1 to 4:1. In aspects, a mole ratio of the reactant aromatic hydrocarbon to the C₆ to C₂₄ straight or branched olefin is 1:1, 2:1, 3:1, or 4:1, or any other value in between the foregoing values.

### Alkylation Catalyst

In aspects, a catalyst is present in the alkylation of the one or more aromatic hydrocarbons with the one or more C₆ to C₂₄ branched or straight olefins. Such a catalyst can be an aluminum halide, an alkyl aluminum halide, a solid acid catalyst (also referred to as an ion exchange resin), an ionic liquid catalyst, a solid super acid (SSA) catalyst, an acidic clay catalyst, boron trifluoride-alcohol catalyst, or combinations thereof. In some aspects, the catalyst can be part of a catalyst system comprising the catalyst.

In aspects, the catalyst can comprise (or consist essentially of, or consist of) an aluminum halide. The halide of the aluminum halide can be fluoride, chloride, bromide, or iodide; alternatively, chloride, bromide, or iodide; alternatively, chloride or bromide; alternatively, chloride or iodide; alternatively, bromide or iodide; alternatively, fluoride; alternatively chloride, or alternatively, bromide. Examples of aluminum halides include, but are not limited to, AlCl₃ and AlBr₃.

In aspects, the catalyst can comprise (or consist essentially of, or consist of) an alkyl aluminum halide compound. In several aspects, the alkyl aluminum catalyst can comprise or can be selected from a dialkyl aluminum halide compound, an alkyl aluminum dihalide compound, an alkyl aluminum sesquihalide compound, or any combination thereof. Generally, each alkyl group of any alkyl aluminum compound described herein, if there is more than one, can independently be a C₁ to C₂₀ alkyl group; alternatively, a C₁ to C₁₁ alkyl group; or alternatively, a C₁ to C₆ alkyl group. In some embodiments, the alkyl group(s) can independently be a methyl group, an ethyl group, a propyl group, a butyl group, a pentyl group, a hexyl group, a heptyl group, or an octyl group; alternatively, a methyl group, an ethyl group, a butyl group, a hexyl group, or an octyl group. In some embodiments, the alkyl group can independently be a methyl group, an ethyl group, an n-propyl group, an n-butyl group, an iso-butyl group, a n-hexyl group, or an n-octyl group; alternatively, a methyl group, an ethyl group, a n-butyl group, or an iso-butyl group; alternatively, a methyl group; alternatively, an ethyl group; alternatively, an n-propyl group; alternatively, an n-butyl group; alternatively, an iso-butyl group; alternatively, an n-hexyl group; or alternatively, an n-octyl group. In several aspects, each aluminum atom in the aluminum alkyl catalyst is connected to three alkyl groups, wherein each alkyl group includes at least two carbon atoms. In several aspects, each aluminum atom in the aluminum alkyl catalyst is connected to three alkyl groups, wherein each alkyl group includes from 2 to 30; alternatively, 2 to 24; alternatively, 2 to 20; or alternatively, 2 to 10 carbon atoms. Exemplary alkyl aluminum halide compounds can include, but are not limited to, diethylaluminum chloride (DEAC), diethylaluminum bromide, ethylaluminum dichloride, ethylaluminum sesquichloride, and mixtures thereof.

In aspects, a solid acid catalyst can comprise (or consist essentially of, or consist of) an acidic ion exchange resin. In embodiments, the solid acid catalyst can comprise (or consist essentially of, or consist of) a styrene-divinylbenzene polymer resin, a functionalized styrene-divinylbenzene polymer resin, a functionalized polymer resin comprising units derived from styrene and units derived from divinyl benzene, a 4-vinylpyridine divinylbenzene polymer resin, an ionomer resin, a tetrafluoroethylene polymer resin modified with perfluorovinyl ether groups terminated with sulfonate groups, or any combination thereof; or alternatively, a styrene-divinylbenzene polymer resin, a functionalized styrene-divinylbenzene polymer resin, a functionalized polymer resin comprising units derived from styrene and units derived from divinyl benzene, or any combination thereof. In yet other embodiment, the solid acid catalyst can comprise (or consist essentially of, or consist of) a styrene-divinylbenzene polymer resin; alternatively, a functionalized styrene-divinylbenzene polymer resin; alternatively, a functionalized polymer resin comprising units derived from styrene and units derived from divinyl benzene; alternatively, a 4-vinylpyridine divinylbenzene polymer resin; alternatively, an ionomer resin; or alternatively, a tetrafluoroethylene polymer resin modified with perfluorovinyl ether groups terminated with sulfonate groups.

A solid acid catalyst can be embodied as a commercially available acidic resin such as an AMBERLYST® resin, a NAFION® resin, or any combination thereof. Various grades of the AMBERLYST® resin and/or the NAFION® resin can be used as the solid acid catalyst. While not limited thereto, the solid acid catalyst can comprise (or consist essentially of, or consist of) AMBERLYST® 15 resin, AMBERLYST® 31 resin, AMBERLYST® 35 resin, AMBERLYST® 36 resin, AMBERLYST® DT resin, or any combination thereof; alternatively, AMBERLYST® 15 resin; alternatively, AMBERLYST® 31 resin; alternatively, AMBERLYST® 35 resin; alternatively, AMBERLYST® 36 resin; or alternatively, AMBERLYST® DT resin.

A solid acid catalyst can be modified or functionalized with an organic acid and/or an inorganic acid; alternatively, an organic acid; or alternatively, an inorganic acid. In some aspects, the solid acid catalyst can be modified with a carboxylic acid, a sulfonic acid, or any combination thereof; alternatively, a carboxylic acid; or alternatively, a sulfonic acid. In aspects, the carboxylic acid can be a C₁ to C₂₀ carboxylic acid; alternatively, a C₁ to C₁₅ carboxylic acid; or alternatively, a C₁ to C₁₁ carboxylic acid. In aspects, the sulfonic acid can be a C₁ to C₂₀ sulfonic acid; alternatively, a C₁ to C₁₅ sulfonic acid; or alternatively, a C₁ to C₁₁ sulfonic acid. In a non-limiting embodiment, the acid which can be utilized to modify the solid acid catalyst can comprise, consist essentially of, or consist of, benzoic acid, formic acid, acetic acid, propionic acid, butyric acid, oxalic acid, trifluoroacetic acid, trichloroacetic acid, sulfamic acid, benzene sulfonic acid, toluene sulfonic acid (ortho, meta, and/or para), dodecylbenzene sulfonic acid, naphthalene sulfonic acid, dinonylnaphthalene disulfonic acid, methane sulfonic acid, or any combination thereof; alternatively, benzoic acid, formic acid, acetic acid, propionic acid, butyric acid, oxalic acid, trifluoroacetic acid, trichloroacetic acid, or any combination thereof; or alternatively, benzene sulfonic acid, toluene sulfonic acid (ortho, meta, and/or para), dodecylbenzene sulfonic acid, naphthalene sulfonic acid, dinonylnaphthalene disulfonic acid, methane sulfonic acid, or any combination thereof. In a non-limiting embodiment, the solid acid catalyst can be modified or functionalized with an acid comprising, consisting essentially of, or consisting of, benzoic acid; alternatively, formic acid; alternatively, acetic acid; alternatively, propionic acid; alternatively, butyric acid; alternatively, oxalic acid; alternatively, trifluoroacetic acid; alternatively, trichloroacetic acid; alternatively, sulfamic acid; alternatively, benzene sulfonic acid; alternatively, toluene sulfonic acid; alternatively, dodecylbenzene sulfonic acid; alternatively, naphthalene sulfonic acid; alternatively, dinonylnaphthalene disulfonic acid; or alternatively, methane sulfonic acid.

In aspects, an ionic liquid catalyst can comprise (or consist essentially of, or consist of) an acidic ionic liquid, or an acidic ionic liquid and a halogenated hydrocarbon; alternatively, an acidic ionic liquid; or alternatively, an acidic ionic liquid and a halogenated hydrocarbon. Acidic ionic liquids and halogenated hydrocarbons are independently described herein and these independent descriptions can be utilized without limitation to further describe any appropriate catalyst system comprising an acid ionic liquid described herein.

Ionic liquids are a category of compounds which are made up entirely of ions and are generally liquids at or below process temperatures. Often, salts which are composed entirely of ions are solids with high melting points, for example, above 450° C. These solids are commonly known as ‘molten salts’ when heated to above their melting points. Sodium chloride, for example, is a common ‘molten salt’, with a melting point of 800° C. Ionic liquids differ from ‘molten salts,’ in that they have low melting points, for example, from −100° C. to 200° C. Ionic liquids tend to be liquids over a very wide temperature range, with some having a liquid range of up to 300° C. or higher. Ionic liquids are generally non-volatile, with effectively no vapor pressure. Many ionic liquids are air stable and water stable, and can be good solvents for a wide variety of inorganic, organic, and polymeric materials.

Ionic liquids can be characterized by the general formula Q⁺A⁻. Generally, Q⁺ gives the ionic liquid a Lewis acidic character. Generally, the mole ratio of A⁻ to Q⁺ can range from 1:1 to 5:1; or alternatively, range from 1:1 to 2:1.

Q⁺ of the ionic liquid can be a quaternary ammonium, quaternary phosphonium, or quaternary sulfonium; alternatively, quaternary ammonium; alternatively, quaternary phosphonium or alternatively, quaternary sulfonium. A⁻ of the ionic liquid can be a negatively charged ion. Negatively charged anions which can be present in ionic liquids include, but are not limited, halides, perhalides, nitrate, tetrahaloborates, hexahalophosphates, hexahaloantinomates, haloaluminates, halotantalates, halocuprates, haloferates, trifluoromethylsulfonium, or any combination thereof; alternatively, chloroaluminates, bromoaluminates, tetrachloroborate, tetrafluoroborate, hexafluorophosphate, trifluoromethane sulfonate, methylsulfonate, p-toluenesulfonate, or any combination thereof; alternatively, chloroaluminates, bromoaluminates, or any combination thereof; alternatively, haloaluminates, or alternatively, bromoalumnates. In some embodiments, the negatively charged ions which can be present in the ionic liquids can include, but are not limited to, Cl⁻, Br⁻, OCl₄⁻, NO₃⁻, BF₄⁻, BCl₄⁻, PF₆⁻, SbF₆⁻, AlCl₄⁻, Al₂Cl₇, AlBr₄⁻, Al₂Br₇, ArF₆⁻, TaF₆⁻, CuCl₂⁻, FeCl₃⁻, ZnCl₃⁻, SO₃CF₃⁻, SO₃Cl₇⁻, or any combination thereof; alternatively, AlCl₄⁻, Al₂Cl₇, AlBr₄⁻, Al₂Br₇, or any combination thereof; alternatively, AlCl₄⁻, Al₂Cl₇, or any combination thereof; or AlBr₄⁻, Al₂Br₇, or any combination thereof. A⁻ which can be used in ionic liquids include, but are not limited to, chloroaluminates, bromoaluminates, tetrachloroborate, tetrafluoroborate, hexafluorophosphate, trifluoromethane sulfonate, methylsulfonate, p-toluenesulfonate. The ionic liquids which can be used advantageously in the present disclosure include acidic haloaluminates; alternatively, chloroaluminates, bromoaluminates, or any combination thereof; alternatively, chloroaluminates; or alternatively, bromoaluminates.

In some embodiments, Q⁺ for the ionic liquid can be amine-based. Among the most common ionic liquids are those formed by reacting a nitrogen-containing heterocyclic ring (cyclic amines), preferably nitrogen-containing aromatic rings (aromatic amines), with an alkylating agent (for example, an alkyl halide) to form a quaternary ammonium salt, followed by ion exchange or other suitable reactions to introduce the appropriate counter anionic species to form ionic liquids. Examples of suitable heteroaromatic rings include pyridine and its derivatives, imidazole and its derivatives, and pyrrole and its derivatives. These rings can be alkylated with varying alkylating agents to incorporate a broad range of alkyl groups on the nitrogen including straight, branched, or cyclic C₁₋₂₀ alkyl group. Frequently, C₁₋₁₂ alkyl groups are used since alkyl groups larger than C₁₂ can produce undesirable solid products with some amines. Pyridinium and imidazolium-based ionic liquids are perhaps the most commonly used ionic liquids. Other amine-based ionic liquids including cyclic and non-cyclic quaternary ammonium salts are frequently used. Phosphonium and sulphonium-based ionic liquids have also been used.

In embodiments, the haloaluminate ionic liquid can be a trialkylammonium haloaluminate ionic liquid, a tetraalkylammonium haloaluminate ionic liquid, hydrogen pyridinium haloaluminate ionic liquid, an N-alkylpryidinium haloaluminate ionic liquid, an N,N′-dialkylimidizolium haloaluminate ionic liquid, or any combination thereof; alternatively, a tetraalkylammonium haloaluminate ionic liquid, an N-alkylpryidinium haloaluminate ionic liquid, an N,N′-dialkylimidizolium haloaluminate ionic liquid, or any combination thereof; alternatively, a tetraalkylammonium haloaluminate ionic liquid; alternatively, an N-alkylpryidinium haloaluminate ionic liquid; or alternatively, an N,N′-dialkylimidizolium haloaluminate ionic liquid.

In embodiments, the haloaluminate ionic liquid can be a chloroaluminate ionic liquid, a bromoaluminate ionic liquid, or any combination thereof; alternatively, a chloroaluminate ionic liquid; or alternatively, a bromoaluminate ionic liquid.

In embodiments, the haloaluminate ionic liquid can be N-(n-butyl)pyridinium chloroaluminate, N-(n-butyl)pyridinium bromoaluminate, or any combination thereof; alternatively, N-(n-butyl)pyridinium bromoaluminate; or alternatively, N-(n-butyl)pyridinium chloroaluminate.

In embodiments, the haloaluminate ionic liquid can have a cationic portion comprising trialkylammonium, tetraalkylammonium, N-alkylpyridinium, or N′,N″-dialkylimidizolium; alternatively, tetraalkylammonium, N-alkylpyridinium, or N′,N″-dialkylimidizolium; alternatively, trialkyl ammonium; alternatively, tetraalkylammonium; alternatively, N-alkylpyridinium; or alternatively, N′,N″-dialkylimidizolium. In embodiments where the cationic portion is trialkylammonium, the cationic portion can have Structure ILC 1. In embodiments where the cationic portion is tetraalkylammonium, the cationic portion can have Structure ILC 2. In embodiments where the cationic portion is N-alkylpyridinium, the cationic portion can have Structure ILC 3 or Structure ILC 4; alternatively, Structure ILC 3; or alternatively, Structure ILC 4. In embodiments where the cationic portion is N′,N″-dialkylimidizolium, the cationic portion can have Structure ILC 5 or Structure ILC 6; alternatively, Structure ILC 5; or alternatively, Structure ILC 6.

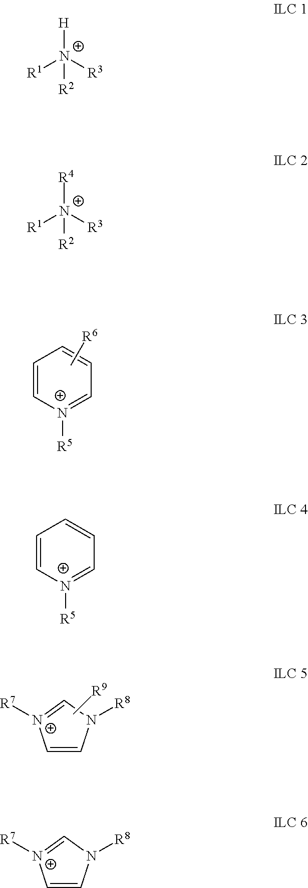

Each R¹, R², and R³ of the trialkylammonium having Structure ILC 1, each R¹, R², R³, and R⁴ of the tetraalkylammonium having Structure ILC 2, each R⁵ and R⁶ of the N-alkylpyridinium having Structure ILC 3, each R⁵ of the N-alkylpyridinium having Structure ILC 4, each R⁷, R⁸, and R⁹ of the N′,N″-dialkylimidizolium having Structure ILC 5, or each R⁷ and R⁸ of the N′,N″-dialkylimidizolium having Structure ILC 6 independently can be a hydrocarbyl group; or alternatively, an alkyl group. General and specific hydrocarbyl groups and alkyl groups are independently described herein as potential substituent groups for various aspects and embodiments described herein and these independently described general and specific hydrocarbyl and alkyl group can be utilized without limitation to further describe each R¹, R², and R³ of the trialkylammonium having Structure ILC 1, each R¹, R², R³, and R⁴ of the tetraalkylammonium having Structure ILC 2, each R⁵ and R⁶ of the N-alkylpyridinium having Structure ILC 3, each R⁵ of the N-alkylpyridinium having Structure ILC 4, each R⁷, R⁸, and R⁹ of the N′,N″-dialkylimidizolium having Structure ILC 5, or each R⁷ and R⁸ of the N′,N″-dialkylimidizolium having Structure ILC 6.

General and specific halogenated hydrocarbons are independently disclosed herein as promoters for Lewis acid catalyst systems. These general and specific halogenated hydrocarbons can be utilized without limitation, and in any combination, with the general and specific ionic liquids disclosed herein to further described catalyst systems comprising, consisting essentially of, or consisting of, (a) an ionic liquid and (b) a halogenated hydrocarbon that can be utilized as the catalyst system comprising an ionic liquid. In embodiments utilizing a haloaluminate ionic liquid, a molar ratio of halide in the halogenated hydrocarbon to aluminum in the haloaluminate ionic liquid can be at least 0.0001:1, 0.001:1, 0.005:1, 0.01:1, 0.025:1, 0.05:1, 0.075:1, 0.1:1, 0.14:1, 0.18:1, or 0.2:1; alternatively or additionally, a maximum molar ratio of halide in the halogenated hydrocarbon to aluminum in the haloaluminate ionic liquid can be less than 10:1, 7.5, 5:1, 4:1, 3:1, 2:1 1.75:1, or 1.5:1. In an embodiment, the molar ratio of halide in the halogenated hydrocarbon to aluminum in the haloaluminate ionic liquid can range from any minimum molar ratio of halide in the halogenated hydrocarbon to aluminum in the haloaluminate ionic liquid to any maximum molar ratio of halide in the halogenated hydrocarbon to aluminum in the haloaluminate ionic liquid described herein. In some embodiments, suitable ranges for the molar ratio of halide in the halogenated hydrocarbon to aluminum in the haloaluminate ionic liquid can include, but are not limited to, a molar ratio of halide in the halogenated hydrocarbon to aluminum in the haloaluminate ionic liquid from 0.0001:1 to 10:1, from 0.001:1 to 7.5:1, from 0.01:1 to 5:1, from 0.025:1 to 5:1, from 0.05:1 to 5:1, from 0.05:1 to 5:1, from 0.1:1 to 5:1, from 0.1:1 to 4:1, from 0.1:1 to 3:1, from 0.12:1 to 4:1, from 0.14:1 to 5:1, from 0.14:1 to 4:1, from 0.14:1 to 3:1, from 0.14:1 to 2:1, from 0.16:1 to 4:1, from 0.16:1 to 3:1, from 0.16:1 to 2:1, from 0.18:1 to 4:1, from 0.18:1 to 3:1, from 0.18:1 to 2:1, from 0.2:1 to 4:1, from 0.2:1 to 3:1, or from 0.2:1 to 2:1. Other suitable molar ratios of halide in the halogenated hydrocarbon to aluminum in the haloaluminate ionic liquid which can be utilized are readily apparent from the present disclosure. The molar ratio of halide in the halogenated hydrocarbon to aluminum in the haloaluminate ionic liquid can be referred to as the halide in the halogenated hydrocarbon to aluminum in the haloaluminate ionic liquid molar ratio.

Additional descriptions of the catalyst systems comprising an acidic ionic liquid suitable for use as a catalyst system in the current disclosure can be found in U.S. Pat. Nos. 10,435,491 and 6,395,948.

In aspects, a solid super acid (SSA) catalyst is a chemically-treated solid oxide, which can also be referred to as a solid oxide treated with an electron-withdrawing anion. In some aspects, the SSA catalyst can include Lewis-acidic metal ion. The SSA catalyst can comprise (or consist essentially of, or consist of) trifluoromethanesulfonic acid (CF₃SO₃H), fluoroantimonic acid (HF:SbF₅), fluorosulfuric acid (HSO₃F), a Brønsted-Lewis acid, a carborane acid (H(CXB₁₁Y₅Z₆) where X, Y, Z═H, Alk, F, Cl, Br, CF₃), anhydrous acid-treated zeolites, fluoroboric acid (HF:BF₃), hydrogen fluoride (HF), perchloric acid (HClO₄), and combinations thereof. In aspects, the Brønsted-Lewis acid is a 1:1 combination of fluorosulfuric acid (HSO₃F) and antimony pentafluoride (SbF₅). Non-limiting examples of suitable SSA catalysts are disclosed in, for instance, U.S. Pat. Nos. 7,294,599, 7,601,665, 7,884,163, 8,309485, 8,623,973, 8,703,886, and 9,023,959.

In aspects, an acidic clay catalyst can comprise (or consist essentially of, or consist of) a natural or synthetic aluminosilicate, including but not limited to smectite, vermiculite, mica, montmorillonite, and combinations thereof. Additionally, positively-charged types of the foregoing types of clays can be treated with various anions. Similarly, negatively charged types of the foregoing types of clays can be treated with various cations. can be modified or functionalized with an organic acid and/or an inorganic acid; alternatively, an organic acid; or alternatively, an inorganic acid. In some aspects, the clay catalyst can be modified with a carboxylic acid, a sulfonic acid, or any combination thereof; alternatively, a carboxylic acid; or alternatively, a sulfonic acid. In aspects, the carboxylic acid can be a C₁ to C₂₀ carboxylic acid; alternatively, a C₁ to C₁₅ carboxylic acid; or alternatively, a C₁ to C₁₁ carboxylic acid. In aspects, the sulfonic acid can be a C₁ to C₂₀ sulfonic acid; alternatively, a C₁ to C₁₅ sulfonic acid; or alternatively, a C₁ to C₁₀ sulfonic acid. In a non-limiting embodiment, the acid which can be utilized to modify the solid acid catalyst can comprise, consist essentially of, or consist of, benzoic acid, formic acid, acetic acid, propionic acid, butyric acid, oxalic acid, trifluoroacetic acid, trichloroacetic acid, sulfamic acid, benzene sulfonic acid, toluene sulfonic acid (ortho, meta, and/or para), dodecylbenzene sulfonic acid, naphthalene sulfonic acid, dinonylnaphthalene disulfonic acid, methane sulfonic acid, or any combination thereof; alternatively, benzoic acid, formic acid, acetic acid, propionic acid, butyric acid, oxalic acid, trifluoroacetic acid, trichloroacetic acid, or any combination thereof; or alternatively, benzene sulfonic acid, toluene sulfonic acid (ortho, meta, and/or para), dodecylbenzene sulfonic acid, naphthalene sulfonic acid, dinonylnaphthalene disulfonic acid, methane sulfonic acid, or any combination thereof. In a non-limiting embodiment, the solid acid catalyst can be modified or functionalized with an acid comprising, consisting essentially of, or consisting of, benzoic acid; alternatively, formic acid; alternatively, acetic acid; alternatively, propionic acid; alternatively, butyric acid; alternatively, oxalic acid; alternatively, trifluoroacetic acid; alternatively, trichloroacetic acid; alternatively, sulfamic acid; alternatively, benzene sulfonic acid; alternatively, toluene sulfonic acid; alternatively, dodecylbenzene sulfonic acid; alternatively, naphthalene sulfonic acid; alternatively, dinonylnaphthalene disulfonic acid; or alternatively, methane sulfonic acid.

In aspects, boron trifluoride-alcohol catalyst can comprise (or consist essentially of, or consist of) a boron trifluoride-methanol.

### Alkylation Product

The alkylation product generally contains one or more types of C₁₆ to C₃₀ alkyl aromatic hydrocarbons, wherein each of the C₁₆ to C₃₀ alkyl aromatic hydrocarbon(s) comprises an aromatic C₆ ring and one, two, or more C₆ to C₂₄ alkyl groups attached to the aromatic C₆ ring. The alkylation product can also include an amount of unreacted reactant aromatic hydrocarbon(s) and an amount of unreacted C₆ to C₂₄ olefin(s).

In aspects, the aromatic C₆ ring can include one or two methyl groups attached thereto. For example, when the reactant aromatic hydrocarbon is toluene, aromatic C₆ rings of the resultant C₁₆ to C₃₀ alkyl aromatic hydrocarbon(s) can have one methyl group attached thereto. Such a methyl group is ortho to, meta to, or para to the attached C₆ to C₂₄ alkyl groups. Similarly, when the reactant aromatic hydrocarbon is xylene, aromatic C₆ rings of the resultant C₂₀ to C₃₀ alkyl aromatic hydrocarbon(s) can have two methyl groups attached thereto, either ortho, meta, or para to one another, and in many cases, the two methyl groups are meta to one another and meta to the attached C₆ to C₂₄ alkyl groups, and in other cases, the two methyl groups are ortho to or meta to one another and/or ortho to or meta to the attached C to C₂₄ alkyl groups.

In aspects, the C₁₆ to C₃₀ alkyl aromatic hydrocarbon comprises a 1,3,5-trisubstituted benzene ring.

In aspects, each of the C₆ to C₂₄ alkyl groups is a branched or straight C₆ straight alkyl group, including hexyl, pentyl, octyl, nonyl, decyl, undecyl, dodecyl, tridecyl, tetradecyl, pentadeccyl, hexadecyl, heptadecyl, octadecyl, nondecyl, heneicosyl, docosyl, tricosyl, and tetracosyl. While each of the C to C₂₄ alkyl groups can be different ones of a branched or straight C₆ straight alkyl group, many times they are the same. In aspects, some or all of the C₆ to C₂₄ alkyl groups are straight C₆ to C₂₄ alkyl groups in which case, the C₆ to C₂₄ olefins from which those C₆ to C₂₄ alkyl groups are derived, can be alpha C₆ to C₂₄ olefins. In aspects, some or all of the C₆ to C₂₄ alkyl groups are branched C₆ to C₂₄ alkyl groups in which case, the C₆ to C₂₄ olefins from which those C₆ to C₂₄ alkyl groups are derived, can be alpha C₆ to C₂₄ olefins or can be C₆ to C₂₄ olefins in which a carbon-carbon double bond is between non-terminal carbons. For example, the optional alkylation catalyst can isomerize an alpha C₆ to C₂₄ olefin to move the carbon-carbon double bond of the olefin from a position between terminal carbon and an adjacent carbon to a position between two adjacent non-terminal carbons, and when the aromatic C₆ ring is alkylated with such a non-alpha olefin, a non-terminal carbon serves as the attachment point to the aromatic C₆ ring.

In aspects, some or all of the aromatic rings of the C₁₆ to C₃₀ alkyl aromatic hydrocarbons has two C₆ to C₁₁ branched or straight alkyl groups attached thereto.

In aspects, some or all of the C₆ to C₂₄ straight or branched alkyl groups are C₁₂ to C₂₄ straight or branched alkyl groups.

In aspects, some or all of the C₆ to C₂₄ alkyl groups is n-tetradecyl or n-hexadecyl.

In aspects, each of the C₁₆ to C₃₀ alkyl aromatic hydrocarbon comprises an aromatic C₆ ring and two hexyl groups and 0-2 methyl groups attached thereto to form a C₁₈ to C₂₀ alkyl aromatic hydrocarbon, including but not limited to a C₁₈ alkyl aromatic hydrocarbon comprising an aromatic C₆ ring and two hexyl groups and 0-2 methyl groups attached thereto, a C₁₉ alkyl aromatic hydrocarbon comprising an aromatic C₆ ring and two hexyl groups and 1 methyl group attached thereto, and a C₂₀ alkyl aromatic hydrocarbon comprising an aromatic C₆ ring and two hexyl groups and 2 methyl groups attached thereto.

In aspects, each of or some of the C₁₆ to C₃₀ alkyl aromatic hydrocarbon comprises an aromatic C₆ ring and one tetracosyl group attached thereto to form a C₂₀ alkyl aromatic hydrocarbon.

In aspects, the alkylation product comprises from about 30 wt % to about 40 wt % of the C₁₆ to C₃₀ alkyl aromatic hydrocarbon based on a total weight of the alkylation product.

Separating Alkylation Product into Reacted and Unreacted Portions (Isolated Portion)

The process can further include separating the alkylation product into a first portion enriched in an amount of unreacted reactant aromatic hydrocarbon and/or an amount of unreacted C₆ to C₂₄ olefin and a second portion enriched in C₁₆ to C₃₀ alkyl aromatic hydrocarbon (the isolated portion). In aspects, the second portion can further include homo-oligomers of the C₆ to C₂₄ olefin. In aspects, the second portion further comprises C₃₀ olefin trimers and C₄₀ olefin tetramers.

Examples of suitable separation techniques for separating the alkylation product can include distillation, fractionation, flashing, stripping, evaporation, or absorption. For example, in distillation, one or more distillation columns can contain trays, baffles, packing, or other structure(s) configured to perform distillation of the alkylation product such that the portion enriched in an amount of unreacted reactant aromatic hydrocarbon emits from the distillation column(s) in a vapor phase and the portion enriched in C₁₆ to C₃₀ alkyl aromatic hydrocarbon emits from the distillation column(s) in a liquid phase.

The unit operations/techniques used for the separating step, such as temperature, pressure, flow rates, and other conditions can be determined by one of ordinary skill in the art.

In aspects, the process can include recycling the unreacted portion to the alkylation reactor where the one or more aromatic hydrocarbons are alkylated with the branched or straight C₆ to C₂₄ olefins.

### Hydrogenating

The process can further include hydrogenating the C₁₆ to C₃₀ alkyl aromatic hydrocarbon(s) to form a hydrogenated product comprising saturated C₁₆ to C₃₀ hydrocarbon(s), where each of the saturated C₁₆ to C₃₀ hydrocarbon(s) comprises a saturated C₆ ring and one or more C₆ to C₂₄ alkyl groups attached to the saturated C₆ ring. Hydrogenation can be accomplished by any means known to those with ordinary skill in the art with the aid of this disclosure. In aspects, all or a portion of the alkylation product, such as a portion enriched in C₁₆ to C₃₀ alkyl aromatic hydrocarbon(s), can be separated from unreacted portions of the reactant aromatic hydrocarbon(s) and C₆ to C₂₄ olefin(s). All of the alkylation production or such a separated portion can be fed to a hydrogenation unit configured to hydrogenate unsaturated double bonds of the aromatic C₆ rings present in the C₁₆ to C₃₀ alkyl aromatic hydrocarbon(s) and to produce a hydrogenated product comprising the C₁₆ to C₃₀ saturated hydrocarbon(s).

Generally, hydrogenation can include contacting the C₁₆ to C₃₀ alkyl aromatic hydrocarbon(s), contained in the alkylation product or the separated portion, and a hydrogenation catalyst under conditions capable of hydrogenating the C₁₆ to C₃₀ alkyl aromatic hydrocarbon(s). In aspects, the hydrogenation catalyst can comprise, or consist essentially of, a supported Group 7, 8, 9, and 10 metals. In some aspects, the hydrogenation catalyst can be selected from the group consisting of one or more of Ni, Pd, Pt, Co, Rh, Fe, Ru, Os, Cr, Mo, and W, supported on silica, alumina, clay, titania, zirconia, or a mixed metal oxide supports. In other aspects, the hydrogenation catalyst can be nickel supported on kieselguhr, platinum or palladium supported on alumina, or cobalt-molybdenum supported on alumina; alternatively, nickel supported on kieselguhr; alternatively, platinum or palladium supported on alumina; or alternatively, cobalt-molybdenum supported on alumina. In yet other aspects, the hydrogenation catalyst can be one or more of the group consisting of nickel supported on kieselguhr, silica, alumina, clay or silica-alumina.

Generally, hydrogenation of the C₁₆ to C₃₀ alkyl aromatic hydrocarbon(s) to form a saturated C₂₀ to C₃₀ hydrocarbon(s) can be performed in any type of process and/or reactor which can hydrogenate the C₁₆ to C₃₀ alkyl aromatic hydrocarbon(s) to have a range of properties disclosed herein for the disclosed saturated C₁₆ to C₃₀ hydrocarbon(s). In an aspect, the hydrogenation of the C₁₆ to C₃₀ alkyl aromatic hydrocarbon(s) to form a saturated C₁₆ to C₃₀ hydrocarbon(s) can be performed in a batch process, a continuous process; or any combination thereof, alternatively a batch process; or alternatively a continuous process. In some aspects, the hydrogenation of the C₁₆ to C₃₀ alkyl aromatic hydrocarbon(s) to form a saturated C₁₆ to C₃₀ hydrocarbon(s) can be performed in a slurry reactor, a continuous stirred tank reactor, a fixed bed reactor or any combination thereof; alternatively, a slurry reactor; alternatively, a continuous stirred tank reactor; or alternatively, a fixed bed reactor. Generally, the hydrogenated product can be filtered to separate the hydrogenation catalyst and/or catalyst fines from the saturated C₁₆ to C₃₀ hydrocarbon(s). Further, the saturated C₁₆ to C₃₀ hydrocarbon(s) can be distilled to further purify the saturated C₁₆ to C₃₀ hydrocarbon(s) into saturated C₁₆ to C₃₀ hydrocarbon fractions; alternatively, distilled to form two or more compositions comprising, or consisting essentially of, saturated C₁₆ to C₃₀ hydrocarbon having different nominal viscosities; or alternatively, distilled to further purify the saturated C₁₆ to C₃₀ hydrocarbons and form two or more compositions comprising, or consisting essentially of, saturated C₁₆ to C₃₀ hydrocarbons having different nominal viscosities.

The quantity of hydrogenation catalyst utilized to hydrogenate the C₁₆ to C₃₀ alkyl aromatic hydrocarbon(s) is dependent upon the identity of the hydrogenation catalyst and the particular hydrogenation process utilized. Generally, the amount of hydrogenation catalyst used can be any amount which can produce the desired saturated C₁₆ to C₃₀ hydrocarbon(s) under the desired conditions capable of forming the saturated C₁₆ to C₃₀ hydrocarbon(s). In a non-fixed bed hydrogenation process (e.g., slurry reactors or continuous stirred tank reactors, among others), the amount of hydrogenation catalyst used in the hydrogenation can range from 0.001 wt % to 20 wt %, 0.01 wt % to 15 wt %, 0.1 wt % to 10 wt %, or 1 wt % to 5 wt % based upon the total weight of the hydrogenation catalyst and the alkylation product or isolated portion being subjected to hydrogenation. In a fixed bed process, the WHSV (weight hourly space velocity) of the alkylation product or separated portion over the hydrogenation catalyst can range from 0.01 to 10, 0.05 to 7.5, or 0.1 to 5.

Generally, the conditions capable of hydrogenating the C₁₆ to C₃₀ alkyl aromatic hydrocarbon(s) can include a hydrogen pressure, a temperature, a contact time, or any combination thereof; alternatively, a hydrogen pressure and a temperature; alternatively, a hydrogen pressure, a temperature, and a contact time. In aspects, the temperature of the hydrogenation that can be utilized as a condition capable of hydrogenating the C₁₆ to C₃₀ alkyl aromatic hydrocarbon(s) can range from 25° C. to 350° C., from 50° C. to 300° C., from 60° C. to 250° C., or from 70° C. to 200° C. In aspects, the hydrogen pressure that can be utilized as a condition capable of hydrogenating the C₁₆ to C₃₀ alkyl aromatic hydrocarbon(s) can range from 100 kPa to 10 MPa, 250 kPa to 7 MPa, 500 kPa to 5 MPa, or 750 kPa to 2 MPa. In aspects, the contact time that can be utilized as a condition capable of hydrogenating the C₁₆ to C₃₀ alkyl aromatic hydrocarbon(s) can range from 1 minutes to 100 hours, from 2 minutes to 50 hours, 5 minutes to 25 hour, or 10 minutes to 10 hours. Additional information and methods for the hydrogenation of olefin oligomers (e.g., olefin oligomer such as the oligomer product that can be produced by the processes described herein) to form polyalphaolefins can be found in U.S. Pat. Nos. 5,573,657 and 10,005,972.

In aspects, greater than 99 wt % of the hydrogenated product is saturated.

### Hydrogenated Product

Hydrogenation of the C₁₆ to C₃₀ alkyl aromatic hydrocarbon(s) in the alkylation product or separated portion results in a hydrogenated product comprising saturated C₁₆ to C₃₀ hydrocarbon(s), each of which comprises a saturated C₆ ring and one, two, or more C₆ to C₂₄ alkyl groups attached to the saturated C₆ ring.

In aspects, the saturated C₆ ring can include one or two methyl groups attached thereto. For example, when the reactant aromatic hydrocarbon is toluene, saturated C₆ rings of the resultant saturated C₁₆ to C₃₀ hydrocarbon(s) can have one methyl group attached thereto. Such a methyl group is ortho to, meta to, or para to the attached C₆ to C₂₄ alkyl groups. Similarly, when the reactant aromatic hydrocarbon is xylene, saturated C₆ rings of the resultant saturated C₁₆ to C₃₀ hydrocarbon(s) can have two methyl groups attached thereto, either ortho, meta, or para to one another, and in many cases, the two methyl groups are meta to one another and meta to the attached C₆ to C₂₄ alkyl groups.

In aspects, each of the C₆ to C₂₄ alkyl groups is a branched or straight C₆ straight alkyl group, including hexyl, pentyl, octyl, nonyl, decyl, undecyl, dodecyl, tridecyl, tetradecyl, pentadeccyl, hexadecyl, heptadecyl, octadecyl, nondecyl, heneicosyl, docosyl, tricosyl, and tetracosyl. While each of the C to C₂₄ alkyl groups can be different ones of a branched or straight C₆ straight alkyl group, many times they are the same. In aspects, some or all of the C₆ to C₂₄ alkyl groups are straight C₆ to C₂₄ alkyl groups in which case, the C₆ to C₂₄ olefins from which those C₆ to C₂₄ alkyl groups are derived, can be alpha C₆ to C₂₄ olefins. In aspects, some or all of the C₆ to C₂₄ alkyl groups are branched C₆ to C₂₄ alkyl groups in which case, the C₆ to C₂₄ olefins from which those C₆ to C₂₄ alkyl groups are derived, can be alpha C₆ to C₂₄ olefins or can be C₆ to C₂₄ olefins in which a carbon-carbon double bond is between non-terminal carbons. For example, the optional alkylation catalyst can isomerize an alpha C₆ to C₂₄ olefin to move the carbon-carbon double bond of the olefin from a position between a terminal carbon and an adjacent carbon to a position between two adjacent non-terminal carbons, and when the aromatic C₆ ring is alkylated with such a non-alpha olefin, a non-terminal carbon serves as the resultant attachment point to the saturated C₆ ring.

In aspects, the saturated C₁₆ to C₃₀ hydrocarbon comprises a 1,3,5-trisubstituted cyclohexane ring.

In aspects, some or all of the saturated C₆ rings of the saturated C₁₆ to C₃₀ hydrocarbons has two C₆ to C₁₀ branched or straight alkyl groups attached thereto.

In aspects, some or all of the C₆ to C₂₄ straight or branched alkyl groups are C₁₂ to C₂₄ straight or branched alkyl groups.

In aspects, some or all of the C₆ to C₂₄ alkyl groups are n-tetradecyl or n-hexadecyl.

In aspects, the hydrogenated product produced by a process described herein can comprise from 85 wt % to 99 wt % of the C₁₆ to C₃₀ saturated hydrocarbon based on a total weight of the hydrogenated product, alternatively, from 86 wt % to 98 wt %, alternatively, from 87 wt % to 97 wt %, alternatively, from 88 wt % to 96 wt %, alternatively, from 89 wt % to 95 wt %, alternatively, from 90 wt % to 94 wt %, alternatively, from 91 wt % to 93 wt %. In some aspects, any hydrogenated product produced by a process herein can comprise the following weight percentages of the C₁₆ to C₃₀ saturated hydrocarbon based on a total weight of the hydrogenated product: 86 wt %, 87 wt %, 88 wt %, 89 wt %, 90 wt %, 91 wt %, 92 wt %, 93 wt %, 94 wt %, 95 wt %, 96 wt %, 97 wt %, or 98 wt % or any value between the foregoing list of values.

The C₁₆ to C₃₀ saturated hydrocarbon exhibits highly satisfactory physical properties, including density, dynamic viscosity, kinematic viscosity, viscosity index, pour point, thermal conductivity, and specific heat. In this regard, the properties of the hydrogenated product in many instances compare favorably with those of commercially available polyalphaolefins.

In aspects, the C₁₆ to C₃₀ saturated hydrocarbon produced by a process described herein can have a density at 100° C. that is in a range of from 0.825 g/cm³ to 0.845 g/cm³, alternatively, from 0.828 g/cm³ to 0.842 g/cm³, alternatively, from 0.830 g/cm³ to 0.840 g/cm³, alternatively, from 0.833 g/cm³ to 0.837 g/cm³, alternatively, from 0.835 g/cm³ to 0.837 g/cm³. In some aspects, the C₁₆ to C₃₀ saturated hydrocarbon produced by a process herein can have a density of 0.830, 0.831, 0.832, 0.833, 0.834, 0.835, 0.836, 0.837, 0.838, 0.839, or 0.840 g/cm³ or any value between the foregoing list of values. Generally, the density can be measured in accordance with ASTM D7042.

In aspects, the C₁₆ to C₃₀ saturated hydrocarbon produced by a process described herein can have a dynamic viscosity at 100° C. that is in a range of from 1.7 to 8 cP (mPa·s), alternatively, from 2 to 7 cP, alternatively, from 2.5 to 6.5 cP, alternatively, from 3 to 6 cP, alternatively, from 3.5 to 5.5 cP, alternatively, from 4 to 6 cP. In some aspects, the C₁₆ to C₃₀ saturated hydrocarbon produced by a process herein can have a dynamic viscosity of 1.7 cP, 2.0 cP, 2.5 cP, 3.0 cP, 3.5 cP, 4.0 cP, 4.5 cP, 5.0 cP, 5.5 cP, 6.0 cP, 6.5 cP, 7.0 cP, 7.5 cP, or 8.0 cP or any value between the foregoing list of values. Generally, the dynamic viscosity at 100° C. can be measured in accordance with ASTM D445 or ASTM D7042.

In aspects, the C₁₆ to C₃₀ saturated hydrocarbon produced by a process described herein can have a dynamic viscosity at 40° C. that is in a range of from 10 to 25 cP (mPa·s), alternatively, from 12 to 23 cP, alternatively, from 14 to 21 cP, alternatively, from 16 to 19 cP. In some aspects, the C₁₆ to C₃₀ saturated hydrocarbon produced by a process herein can have a dynamic viscosity of 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, or 25 cP or any value between the foregoing list of values. Generally, the dynamic viscosity at 40° C. can be measured in accordance with ASTM D445 or ASTM D7042.

In aspects, the C₁₆ to C₃₀ saturated hydrocarbon produced by a process described herein can have a kinematic viscosity at 100° C. that is in a range of from 1.7 to 8 cSt (mm²/s), alternatively, from 2 to 7 cSt, alternatively, from 2.5 to 6.5 cSt, alternatively, from cSt, alternatively, from 3.5 to 5.5 cSt, alternatively, from 4 to 6 cSt. In some aspects, the C₁₆ to C₃₀ saturated hydrocarbon produced by a process herein can have a kinematic viscosity of 1.7 cSt, 2 cSt, 2.5 cSt, 3 cSt, 3.5 cSt, 4 cSt, 4.5 cSt, 5 cSt, 5.5 cSt, 6 cSt, 6.5 cSt, 7 cSt, 7.5 cSt, or 8 cSt or any value between the foregoing list of values. Generally, the kinematic viscosity at 100° C. can be measured in accordance with ASTM D445 or ASTM D7042.

In aspects, the C₁₆ to C₃₀ saturated hydrocarbon produced by a process described herein can have a kinematic viscosity at 40° C. that is in a range of from 10 to 25 cSt (mm²/s), alternatively, from 12 to 23 cSt, alternatively, from 14 to 21 cSt, alternatively, from 16 to 19 cSt. In some aspects, the C₁₆ to C₃₀ saturated hydrocarbon produced by a process herein can have a dynamic viscosity of 10 cSt, 11 cSt, 12 cSt, 13 cSt, 14 cSt, 15 cSt, 16 cSt, 17 cSt, 18 cSt, 19 cSt, 20 cSt, 21 cSt, 22 cSt, 23 cSt, 24 cSt, or 25 cSt or any value between the foregoing list of values. Generally, the kinematic viscosity at 40° C. can be measured in accordance with ASTM D445 or ASTM D7042.

In aspects, the C₁₆ to C₃₀ saturated hydrocarbon produced by a process described herein can have a viscosity index in a range of from 80 to 130, alternatively, from 85 to 125, alternatively, from 90 to 120, alternatively, from 95 to 115, alternatively, from 100 to 110. In some aspects, the C₁₆ to C₃₀ saturated hydrocarbon produced by a process herein can have a viscosity index of 80° C., 85° C., 90° C., 95° C., 98.2° C., 100° C., 105° C., 106.2° C., 110° C., 115° C., 119.8° C., 120° C., 125° C., or 130° C. or any value between the foregoing list of values. Generally, the viscosity index can be measured in accordance with ASTM D2270.

In aspects, the C₁₆ to C₃₀ saturated hydrocarbon produced by a process described herein can have a pour point in a range of from −20° C. to −60° C., alternatively, from −24° C. to −56° C., alternatively, from −28° C. to −52° C., alternatively, from −32° C. to −48° C., alternatively, from −36° C. to −44° C., alternatively, from −39° C. to −44° C. In some aspects, the C₁₆ to C₃₀ saturated hydrocarbon produced by a process herein can have a pour point of −20° C., −25° C., −30° C., −35° C., −40° C., −45° C., −50° C., −55° C., −60° C., or any value between the foregoing list of values. Generally, the pour point can be measured in accordance with ASTM D5950.

In aspects, any hydrogenated product produced by a process described herein can have a thermal conductivity at 40° C. in a range of from 125 to 135 mW/(m·K); alternatively, from 126 to 134 mW/(m·K); alternatively, from 127 to 133 mW/(m·K); alternatively, from 128 to 132 mW/(m·K); alternatively, from 129 to 131 mW/(m·K); alternatively, from 130 to 131 mW/(m·K). In some aspects, any hydrogenated product produced by a process described herein can have a thermal conductivity at 40° C. of 130.0, 130.1, 130.2, 130.3, 130.4, 130.5, 130.6, 130.7, 130.8, 130.9, 131.0 mW/(m·K), or any value between the foregoing list of values. Thermal conductivity is measured in accordance with ASTM D7896-19.

In aspects, any hydrogenated product produced by a process described herein can have a specific heat at 30° C. in a range of from 1890.0 to 1910.0 J/(kg·K); alternatively, from 1895.0 to 1905.0 J/(kg·K). In some aspects, any hydrogenated product produced by a process described herein can have specific heat at 30° C. of 1895.0, 1896.0, 1897.0, 1898.0, 1899.0, 1900.0, 1901.0, 1902.0, 1903.0, 1904.0, or 1905.0 J/(kg·K), or any value between the foregoing list of values. Specific heat is measured in accordance with ASTM D7896-19.

In aspects, the saturated hydrocarbons, also which can be referred to as alkyl-substituted cyclohexanes described herein can be used in a variety of components or products for a diverse range of applications and industries. For example, they can be used as a lubrication fluid (or a component of a lubrication fluid). Exemplary lubrication fluids in which the saturated alkyl cyclohexanes produced by the processes described herein can be utilized include, but are not limited to, automotive lubricants, crank case lubricants, driveline lubrication fluids, gear lubricants, greases, gearbox oils, engine oils, transmission fluids, and/or drilling fluids. Exemplary functional fluids in which the polyalphaolefins produced by the processes described herein can be utilized include, but are not limited to, hydraulic fluids, drilling fluids, fracturing fluids, thermal management fluids, metal working fluids, coolant fluids, dielectric coolant fluids (e.g., for immersion cooling of electronic and/or computer devices).

### Immersion Coolant and Cooling Process

In aspects, the hydrogenated product can be utilized as coolant fluid for equipment needing cooling. In such uses, the equipment to be cooled in placed in direct heat transfer relationship with a bath or recirculating flow of the coolant fluid to remove heat therefrom. Heat from the coolant fluid can be removed through heat exchange with another heat exchange fluid, such as in the case of a static immersion bath, or can be removed through heat exchange with the ambient environment with use of a heat exchanger such as a fin fan heat exchanger. In aspects, the equipment to be cooled is a computer, a computer server, a battery, and/or a motor.

### EXAMPLES

The following examples are illustrative of the alkylation of an aromatic compound with a C₆ to C₂₄ olefin to produce an alkylation product comprising a C₁₆ to C₃₀ alkyl aromatic hydrocarbon, followed by hydrogenation of the C₁₆ to C₃₀ alkyl aromatic hydrocarbon to form a C₁₆ to C₃₀ saturated hydrocarbon.

### Example 1

In Example 1, the aromatic compound was m-xylene, the C₆ to C₂₄ olefin was 1-tetradecene, the C₁₆ to C₃₀ alkyl aromatic hydrocarbon was a C₂₂ alkyl aromatic hydrocarbon, and the C₁₆ to C₃₀ saturated hydrocarbon was a C₂₂ saturated hydrocarbon having a saturated C₆ ring, a C₁₄ alkyl group attached at the 1 position on the saturated C₆ ring, and two methyl groups attached to the 3 and 5 positions on the saturated C₆ ring, forming a 1,3,5-trisubstituted cyclohexane ring.

An amount of 530 g, corresponding to 5.0 mol, of m-xylene was combined with 1.3 g, corresponding to 10 mmol, of AlCl₃ catalyst in a 1.0 liter, three-necked flask under a nitrogen purge. An amount of 196 g, corresponding to 1.0 mol, of 1-tetradecene was added to the flask over a course of 18 h, producing a maximum isotherm of less than 30° C. The reaction mixture was quenched with 10 g NaOH dissolved in 50 ml of water.

GC analysis of the alkylation product showed essentially complete conversion of the 1-tetradecene to alkyl groups on the aromatic ring. It additionally showed that no dimer of the 1-tetradecene was present. While the GC separation was not performed long enough for a dialkyl aromatic hydrocarbon to elute, it did show an alkylation reaction yield, of 92% based on the limiting reagent, for the C₂₂ (mono) alkyl aromatic hydrocarbon.

The alkylation product was then heated with stirring at 70° C. overnight to remove the remaining m-xylene and produce an isolated product. After hydrogenation of the isolated product, various physical properties of the resultant C₂₂ saturated alkyl cyclohexane product (including C₂₂ saturated alkyl cyclohexane) were measured. The resulting C₂₂ saturated alkyl cyclohexane product had a density at 15° C. of 0.8366 g/cm³, a kinematic viscosity at 40° C. of 12.54 cSt (mm²/s), a kinematic viscosity at 100° C. of 3.05 cSt (mm²/s), a viscosity index of 98.2, a dynamic viscosity at 40° C. of 10.29 cP (mPa·s), a dynamic viscosity at 100° C. of 2.38 cP (mPa·s), and a pour point of −50.0° C.

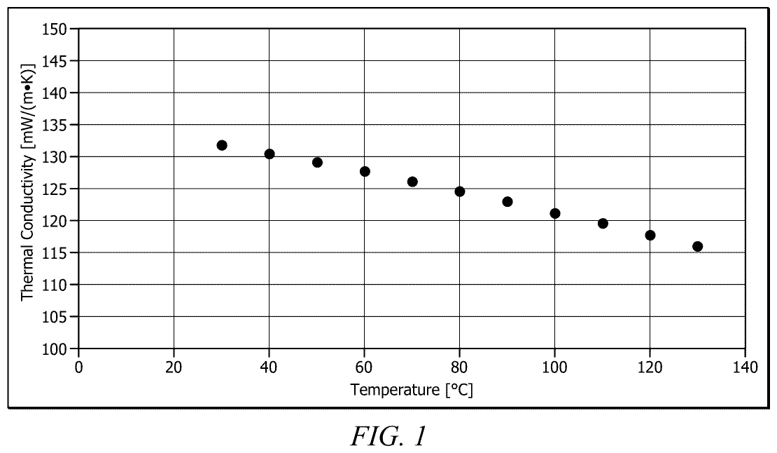

The thermal conductivity of the hydrogenated product was measured over a wide range of temperatures. A plot of the measurements, in mW/(m·K) vs ° C., is shown in FIG. 1. The thermal conductivity decreases smoothly as the temperature increases and compares favorably to polyalphaolefins as a coolant.

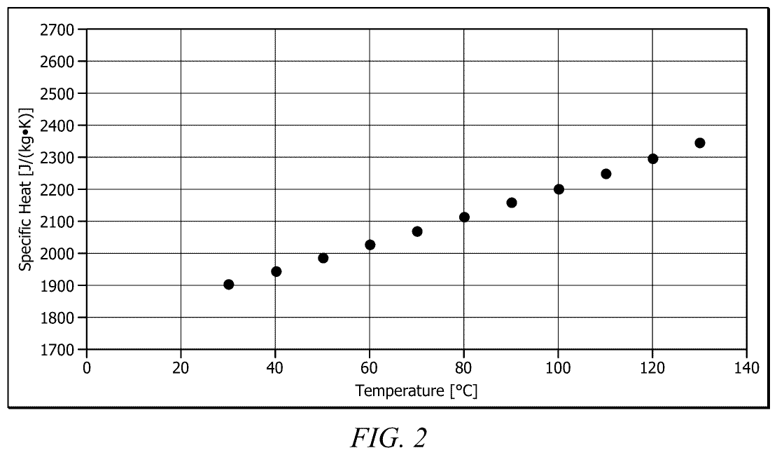

The specific heat of the hydrogenated product was also measured over a wide range of temperatures. A plot of the measurements, J/kg-K vs ° C., is shown in FIG. 2. The specific heat increases smoothly as the temperature increases and the values compare favorably to those of polyalphaolefins used as a coolant.

### Example 2

In Example 2, the aromatic compound was m-xylene, the C₆ to C₂₄ olefin was 1-hexadecene, the C₁₆ to C₃₀ alkyl aromatic hydrocarbon was a C₂₄ alkyl aromatic hydrocarbon, and the C₁₆ to C₃₀ saturated hydrocarbon was a C₂₄ saturated hydrocarbon having a saturated C₆ ring, a C₁₆ alkyl group attached at the 1 position on the saturated C₆ ring, and two methyl groups attached to the 3 and 5 positions on the saturated C₆ ring, forming a 1,3,5-trisubstituted cyclohexane ring.

An amount of 371 g, corresponding to 3.5 mol, of m-xylene was combined with 1.0 g of AlCl₃ in a reaction vessel, which remained under a nitrogen purge gas. An amount of 198 g, corresponding to 0.9 mol, of 1-hexadecene was slowly added to the reaction vessel. This corresponds to a 4:1 mole ratio of m-xylene:1-hexadecene. The reaction, which produced an exotherm peak of 36° C., was completed in about 5 hr. The reaction mixture was then stirred for an additional 2 hr before being quenched with addition of 100 ml of a 5% NaOH solution.

Quantitative analysis via gas chromatography indicated that 98% of the 1-hexadecene reactant was converted to an alkylate group on the aromatic ring. It further indicated that a C₂₄ alkyl aromatic hydrocarbon was present in the alkylation product at a concentration of 91 wt %. Excluding the presence of the 1-hexadecene reactant not attached to the aromatic ring, the C₂₄ alkyl aromatic hydrocarbon was present at a concentration of 93 wt %.

The alkylation product was subjected to distillation, yielding an isolated C₂₄ alkyl aromatic hydrocarbon. After hydrogenation of the isolated product, various physical properties of the resulting saturated alkyl cyclohexane product (including C₂₄ saturated alkyl cyclohexane) were measured, yielding a kinematic viscosity at 40° C. of 19.04 cSt, a kinematic viscosity at 100° C. of 4.13 cSt, a viscosity index of 119.8, and a pour point of −23° C.

### Example 3

In Example 3, the aromatic compound was m-xylene, the C₆ to C₂₄ olefin was 1-hexadecene, the C₁₆ to C₃₀ alkyl aromatic hydrocarbon was a C₂₄ alkyl aromatic hydrocarbon, and the C₁₆ to C₃₀ saturated hydrocarbon was a C₂₄ saturated hydrocarbon having a saturated C₆ ring, a C₁₆ alkyl group attached at the 1 position on the saturated C₆ ring, and two methyl groups attached to the 3 and 5 positions on the saturated C₆ ring, forming a 1,3,5-trisubstituted cyclohexane ring.

An amount of 318 g, corresponding to 3.0 mol, of m-xylene was combined with 35 g of Amberlyst A-15 catalyst in a reaction vessel, which remained under a nitrogen purge gas. An amount of 224 g, corresponding to 1.0 mol, of 1-hexadecene was slowly added to the reaction vessel. This corresponds to a 3:1 ratio of m-xylene:1-hexadecene. The reaction mixture was then stirred for an additional 3 hr.

Quantitative analysis via gas chromatography indicated that a C₂₄ alkyl aromatic hydrocarbon was present in the alkylation product at a concentration of 89 wt %. Excluding the presence of unconsumed 1-hexadecene reactant not attached as an alkyl group to the aromatic ring, the C₂₄ alkyl aromatic hydrocarbon was present at a concentration of 90 wt %. Utilization of a different GC method that separates the C₂₄ alkyl aromatic hydrocarbon from 1-hexadecene indicated that 98 wt % of the m-xylene was alkylated with only 2 wt % of the non-alkylated m-xylene remaining.

The alkylation product was then stirred overnight, filtered, and distilled to yield a C₂₄ alkyl aromatic hydrocarbon isolate. After hydrogenation of the isolated product, various physical properties of the resulting C₂₄ saturated alkyl cyclohexane product were measured. The C₂₄ saturated alkyl cyclohexane product had a kinematic viscosity at 40° C. of 15.53 cSt, a kinematic viscosity at 100° C. of 3.53 cSt, a viscosity index of 106.2, and a pour point of −33.0° C.

In comparison to the C₂₄ saturated alkyl cyclohexane product produced using the Amberlyst A-15 catalyst, the C₂₄ saturated alkyl cyclohexane product produced using the AlCl₃ catalyst resulted in higher viscosity, higher viscosity index, and lower pour point of the C₂₄ saturated alkyl cyclohexane product.

### Additional Disclosure

Accordingly, the scope of protection is not limited by the description set out above but is only limited by the claims which follow, that scope including all equivalents of the subject matter of the claims. Each and every claim is incorporated into the specification as part of the disclosure. Thus, the claims are a further description and are an addition to the detailed description. The disclosures of all patents, patent applications, and publications cited herein are hereby incorporated by reference.

Aspect 1. A process comprising: alkylating i) a reactant aromatic hydrocarbon comprising benzene, toluene, xylene, or combinations thereof with ii) a C₆ to C₂₄ olefin, to produce an alkylation product comprising a C₁₆ to C₃₀ alkyl aromatic hydrocarbon, wherein the C₁₆ to C₃₀ alkyl aromatic hydrocarbon comprises an aromatic C₆ ring and one or more C₆ to C₂₄ alkyl groups attached to the aromatic C₆ ring; and hydrogenating the C₁₆ to C₃₀ alkyl aromatic hydrocarbon to produce a hydrogenated product comprising a C₁₆ to C₃₀ saturated hydrocarbon, wherein the C₁₆ to C₃₀ saturated hydrocarbon comprises a saturated C₆ ring and one or more C₆ to C₂₄ alkyl groups attached to the saturated C₆ ring.

Aspect 2. The process of Aspect 1, wherein the hydrogenated product has a density at 15° C. in a range of from 0.825 g/cm3 to about 0.845 g/cm3 when measured in accordance with ASTM D7042.

Aspect 3. The process of Aspect 1 or 2, wherein the hydrogenated product has a pour point in a range of from −20° C. to −60° C. when measured in accordance with ASTM D5950, a dynamic viscosity in a range of from 1.7 to 8 cP when measured at 100° C. in accordance with ASTM D7042, a kinematic viscosity in a range of from 1.7 to 8 cSt when measured at 100° C. in accordance with ASTM D7042 or ASTM D445.

Aspect 4. The process of any one of Aspects 1 to 3, wherein greater than 99 wt % of the hydrogenated product is saturated.

Aspect 5. The process of any one of Aspects 1 to 4, wherein the hydrogenated product comprises from about 85 wt % to about 99 wt % of the C₁₆ to C₃₀ saturated hydrocarbon based on a total weight of the hydrogenated product.

Aspect 6. The process of Aspect 1, wherein the C₆ to C₂₄ olefin comprises a C₆ to C₁₁ linear alpha olefin, wherein the saturated C₆ ring has two C₆ to C₁₁ alkyl groups attached to the saturated C₆ ring.

Aspect 7. The process of Aspect 1, wherein the C₆ to C₂₄ olefin is branched.

Aspect 8. The process of Aspect 1, wherein the C₆ to C₂₄ olefin comprises a C₁₂ to C₂₄ olefin, wherein the saturated C₆ ring has one C₁₂ to C₂₄ alkyl group attached to the saturated C₆ ring.

Aspect 9. The process of Aspect 8, wherein the C₁₂ to C₂₄ olefin is a branched internal olefin.

Aspect 10. The process of any one of Aspects 1 to 9, wherein the alkylation product comprises from about 30 wt % to about 40 wt % of the C₁₆ to C₃₀ alkyl aromatic hydrocarbon based on a total weight of the alkylation product.

Aspect 11. The process of Aspect 10, wherein the alkylation product further comprises unreacted aromatic hydrocarbon, the process further comprising: separating the alkylation product into a first portion comprising the unreacted aromatic hydrocarbon and a second portion comprising the C₁₆ to C₃₀ alkyl aromatic hydrocarbon.

Aspect 12. The process of Aspect 11, wherein the second portion further comprises C₃₀ olefin trimers and C₄₀ olefin tetramers, wherein the C₃₀ olefin trimers and C₄₀ olefin tetramers are hydrogenated with the C₁₆ to C₃₀ alkyl aromatic hydrocarbon.

Aspect 13. The process of any one of Aspects 1 to 12, wherein alkylating is performed in a presence of a catalyst comprising an aluminum halide catalyst.

Aspect 14. The process of any one of Aspects 1 to 13, wherein during alkylating, a mole ratio of the reactant aromatic hydrocarbon to the C₆ to C₂₄ olefin is in a range of from 1:1 to excess:1.

Aspect 15. The process of any one of Aspects 1 to 14, where the reactant aromatic hydrocarbon comprises m-xylene.

Aspect 16. The process of Aspect 15, wherein the C₁₆ to C₃₀ alkyl aromatic hydrocarbon comprises a 1,3,5-trisubstituted benzene ring, wherein the C₁₆ to C₃₀ saturated hydrocarbon comprises a 1,3,5-trisubstituted cyclohexane ring.

Aspect 17. An alkyl cyclohexane comprising a C₁₆ to C₃₀ saturated hydrocarbon, wherein the C₁₆ to C₃₀ saturated hydrocarbon comprises a saturated C₆ ring and one or more C₆ to C₂₄ alkyl groups attached to the saturated C₆ ring.

Aspect 18. The alkyl cyclohexane of Aspect 17, having a density at 15° C. in a range of from 0.825 g/cm3 to 0.845 g/cm3 when measured in accordance with ASTM D7042, a pour point in a range of from −20° C. to −60° C. when measured in accordance with ASTM D5950, a dynamic viscosity in a range of from 2 to 8 cP when measured at 100° C. in accordance with ASTM D7042, a kinematic viscosity in a range of from 2 to 8 cSt when measured at 100° C. in accordance with ASTM D7042 or ASTM D445.

Aspect 19. An immersion coolant comprising the alkyl cyclohexane of Aspect 17.

Aspect 20. A process comprising: cooling an equipment with an immersion coolant comprising the alkyl cyclohexane of Aspect 17.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein can be utilized according to the present disclosure. Accordingly, the appended claims are intended to include within their scope such processes, machines, manufacture, compositions of matter, means, methods, or steps.

What is claimed is:

1. A process comprising:
alkylating i) a reactant aromatic hydrocarbon comprising benzene, toluene, xylene, or combinations thereof with ii) a C₆ to C₂₄ olefin, to produce an alkylation product comprising a C₁₆ to C₃₀ alkyl aromatic hydrocarbon, wherein the C₁₆ to C₃₀ alkyl aromatic hydrocarbon comprises an aromatic C₆ ring and one or more C₆ to C₂₄ alkyl groups attached to the aromatic C₆ ring; and
hydrogenating the C₁₆ to C₃₀ alkyl aromatic hydrocarbon to produce a hydrogenated product comprising a C₁₆ to C₃₀ saturated hydrocarbon, wherein the C₁₆ to C₃₀ saturated hydrocarbon comprises a saturated C₆ ring and one or more C₆ to C₂₄ alkyl groups attached to the saturated C₆ ring.

2. The process of claim 1, wherein the hydrogenated product has a density at 15° C. in a range of from 0.825 g/cm³ to about 0.845 g/cm³ when measured in accordance with ASTM D7042.

3. The process of claim 2, wherein the hydrogenated product has a pour point in a range of from −20° C. to −60° C. when measured in accordance with ASTM D5950, a dynamic viscosity in a range of from 1.7 to 8 cP when measured at 100° C. in accordance with ASTM D7042, a kinematic viscosity in a range of from 1.7 to 8 cSt when measured at 100° C. in accordance with ASTM D7042 or ASTM D445.

4. The process of claim 2, wherein greater than 99 wt % of the hydrogenated product is saturated.

5. The process of claim 1, wherein the hydrogenated product comprises from about 85 wt % to about 99 wt % of the C₁₆ to C₃₀ saturated hydrocarbon based on a total weight of the hydrogenated product.

6. The process of claim 1, wherein the C₆ to C₂₄ olefin comprises a C₆ to C₁₀ linear alpha olefin, wherein the saturated C₆ ring has two C₆ to C₁₁ alkyl groups attached to the saturated C₆ ring.

7. The process of claim 1, wherein the C₆ to C₂₄ olefin is branched.

8. The process of claim 1, wherein the C₆ to C₂₄ olefin comprises a C₁₂ to C₂₄ olefin, wherein the saturated C₆ ring has one C₁₂ to C₂₄ alkyl group attached to the saturated C₆ ring.

9. The process of claim 8, wherein the C₁₂ to C₂₄ olefin is a branched internal olefin.

10. The process of claim 1, wherein the alkylation product comprises from about 30 wt % to about 40 wt % of the C₁₆ to C₃₀ alkyl aromatic hydrocarbon based on a total weight of the alkylation product.

11. The process of claim 10, wherein the alkylation product further comprises unreacted aromatic hydrocarbon, the process further comprising:
separating the alkylation product into a first portion comprising the unreacted aromatic hydrocarbon and a second portion comprising the C₁₆ to C₃₀ alkyl aromatic hydrocarbon.

12. The process of claim 11, wherein the second portion further comprises C₃₀ olefin trimers and C₄₀ olefin tetramers, wherein the C₃₀ olefin trimers and C₄₀ olefin tetramers are hydrogenated with the C₁₆ to C₃₀ alkyl aromatic hydrocarbon.

13. The process of claim 1, wherein alkylating is performed in a presence of a catalyst comprising an aluminum halide catalyst.

14. The process of claim 1, wherein during alkylating, a mole ratio of the reactant aromatic hydrocarbon to the C₆ to C₂₄ olefin is in a range of from 1:1 to excess:1.

15. The process of claim 1, where the reactant aromatic hydrocarbon comprises m-xylene.

16. The process of claim 15, wherein the C₁₆ to C₃₀ alkyl aromatic hydrocarbon comprises a 1,3,5-trisubstituted benzene ring, wherein the C₁₆ to C₃₀ saturated hydrocarbon comprises a 1,3,5-trisubstituted cyclohexane ring.

17. An alkyl cyclohexane comprising a C₁₆ to C₃₀ saturated hydrocarbon, wherein the C₁₆ to C₃₀ saturated hydrocarbon comprises a saturated C₆ ring and one or more C₆ to C₂₄ alkyl groups attached to the saturated C₆ ring.

18. The alkyl cyclohexane of claim 17, having a density at 15° C. in a range of from 0.825 g/cm³ to 0.845 g/cm³ when measured in accordance with ASTM D7042, a pour point in a range of from −20° C. to −60° C. when measured in accordance with ASTM D5950, a dynamic viscosity in a range of from 2 to 8 cP when measured at 100° C. in accordance with ASTM D7042, a kinematic viscosity in a range of from 2 to 8 cSt when measured at 100° C. in accordance with ASTM D7042 or ASTM D445.

19. An immersion coolant comprising the alkyl cyclohexane of claim 17.

20. A process comprising:
cooling an equipment with an immersion coolant comprising the alkyl cyclohexane of claim 17.
